(19) United States
(12) Patent Application Publication
(10) Pub. No.: US 2020/0297918 A1
(43) Pub. Date: Sep. 24, 2020

(54) AN INJECTION DEVICE WITH A CLEANING RESERVOIR

(71) Applicant: Novo Nordisk A/S, Bagsvaerd (DK)

(72) Inventors: Bo Radmer, Hilleroed (DK); Lars Eilertsen, Fredensborg (DK); Bjoern Gullak Larsen, Birkeroed (DK); Nikolaj Thorsoe Kronborg, Koebenhavn (DK)

(21) Appl. No.: 16/649,926

(22) PCT Filed: Sep. 10, 2018
(86) PCT No.: PCT/EP2018/074281

§ 371 Date: Mar. 23, 2020

(30) Foreign Application Priority Data
Sep. 25, 2017 (EP) 17192806.2

(51) Int. Cl. A61M 5/00; A61M 5/32

(52) U.S. Cl. CPC A61M 5/001; A61M 5/3204; A61M 2209/10; A61M 5/326; A61M 5/3245

(57) ABSTRACT

The invention relates to a medical injection device for injection of a liquid drug. The injection device comprises a housing structure with a cartridge and needle cannula. Further an axially movable cleaning reservoir is provided which is movable in relation to the housing structure, and which cleaning reservoir contains a liquid cleaning agent. The distal tip of the needle cannula is positioned and thus cleaned inside the cleaning reservoir between subsequent injections. According to the invention, the cleaning reservoir connects to an overflow reservoir through a one-way pressure valve such that the cleaning agent and/or air is flowable from the cleaning reservoir and into the overflow reservoir.

### THE TECHNICAL FIELD OF THE INVENTION

The present invention relates to an injection device having a cleaning reservoir for cleaning the distal tip of the needle cannula between subsequent injections. The invention especially refers to such injection device wherein the cleaning reservoir is movable in an axial direction between a cleaning position and a position allowing injection.

### DESCRIPTION OF RELATED ART

Injection devices wherein the needle cannula is covered by a telescopically movable needle shield during injection are widely known. In some of the more recent of these injection devices, the telescopically movable needle shield carries a cleaning reservoir which cleans the distal tip of the needle cannula between subsequent injections. WO 2015/062845, WO2015/173151, WO2017/032599 and WO2017/050694 provide examples of such injection devices.

In the injection device disclosed in WO 2015/062845, a volume of the preservative containing liquid drug contained in the cartridge of the injection device is filled into the cleaning reservoir to be used as a cleaning agent for cleaning the injection tip of the needle cannula between injections. In order to allow air trapped in the cleaning reservoir to escape as the preservative containing liquid drug is being filled in to the cleaning reservoir one of the two septum sealing the cleaning reservoir are provided with a venting area through which the trapped air can escape.

WO2015/173151 discloses a medical injection device wherein the cleaning reservoir is provided with an opening which connects to an overflow reservoir such that any volume expansion of the liquid cleaning solvent inside the cleaning reservoir can flow into the overflow reservoir. Due to the opening the liquid cleaning solvent flows back into the cleaning reservoir when the volume of the liquid cleaning solvent decreases.

In WO 2017/032599 it is suggested to provide the cleaning reservoir with a valve to let the air out as the preservative containing liquid drug flows into the cleaning reservoir. Different examples of such valves are provided in WO 2017/050694.

No matter if the cleaning reservoir is to be filled with the same preservative containing drug as present in the cartridge or is to filled with a different liquid cleaning agent it is always necessary to remove the air inside the cleaning reservoir as the cleaning reservoir is being filled and preferably to seal the cleaning reservoir once the reservoir has been filled

### DESCRIPTION OF THE INVENTION

The valves described in the prior art are all mechanical on-off valves which requires the user to actively operate the valve in order to close the valve and thus seal the cleaning chamber.

As the user occasionally forgets to activate the mechanical valve it is an object of the present invention to provide a valve which does not depend on the memory of the user.

Accordingly, in one aspect, the present invention relates to a medical injection device for injection of a liquid drug. In a first example the injection device comprises:
A housing structure supporting or securing a cartridge. The cartridge contains the liquid drug to be injected and comprises a plunger which is movable preferably in a distal direction to thereby pressurize the liquid drug contained in the cartridge.
A needle cannula having a distal part and a proximal part. The distal part has a distal tip and the proximal part connects to the cartridge at least during injection. The needle cannula is mounted relatively to the housing structure such that the distal tip extends in a distal direction and wherein a hollow longitudinal lumen stretches along a centre line.
An axially movable cleaning reservoir which is movable in relation to the housing structure. The cleaning reservoir contains a liquid cleaning agent and the distal tip of the needle cannula is positioned inside the cleaning reservoir at least between injections.

According to the present invention an overflow reservoir connects to the cleaning reservoir through a one-way pressure valve which comprises a flexible element changeable from a default closed state to an activated open state and vice versa.

Further, and according to the present invention, the flexible element is changeable from the default closed state and into the activated open state when the pressure inside the cleaning chamber surpasses a predetermined level. In the default closed state the flexible element seals off the cleaning reservoir and in the activated open state the flexible element allow the cleaning agent and/or air to flow from the cleaning reservoir and into the overflow reservoir.

Due to various tolerances it often happens that the amount of the liquid cleaning agent filled into the cleaning reservoir has a volume larger than the physical volume of the cleaning reservoir. In such case it is very beneficial if the extra volume is able to escape to thereby avoid that the cleaning reservoir is pressurized. Further, it is necessary that the air inside the cleaning reservoir can escape as the cleaning reservoir is being filled.

For these purposes an overflow reservoir is provided and in order to prevent the escaped liquid cleaning agent and/or air from flowing back into the cleaning reservoir, the two chambers are separated by a one-way pressure valve.

The one-way pressure valve is shaped such that the pressure of the liquid cleaning agent and/or air automatically opens the one-way pressure valve when the pressure has reached a certain level. The one-way pressure valve also automatically closes once the pressure has fallen below this level.

This is preferably achieved by making at a part of the one-way valve from a flexible member which is changeable from a default closed state to an activated open state in response to the pressure inside the cleaning reservoir.

Once this pressure surpasses a predetermined value the flexible member opens automatically in response to the pressure build up and thus allow liquid cleaning agent and/or air to flow into the overflow reservoir. Once the pressure falls below this predetermined level again the flexible member resumes its initial closed state and seals off the cleaning reservoir.

The one-way valve is thus opened and closed in response to the pressure inside the cleaning reservoir and does henceforth not rely on a user manually opening and closing the valve. Further, the one-way pressure valve prevents backflow from the overflow reservoir and back into the cleaning reservoir as the one-way pressure valve only opens in response to an overpressure inside the cleaning reservoir

The medical injection device is in a further example provided with an axially movable needle shield which is movable between a first position and a second position.

In the first position, the needle shield covers the distal tip of the needle cannula. By covers is only meant that the movable needle shield radially conceals the needle cannula. In the axial direction the movable needle shield is slidable to move to a position wherein the distal tip of the needle cannula is in front of the movable needle shield. The latter position of the movable needle shield thus being the second position.

The movable needle shield preferably carries the cleaning reservoir, such that the distal tip of the needle cannula is positioned inside the cleaning reservoir when the needle shield is in the first position which is usually between injections.

In the second position, which is usually during injection, the distal tip of the needle cannula extends distally in relation to the cleaning reservoir.

The cleaning reservoir and the overflow reservoir preferably connect through a channel. This channel can be radial or axial or any combination thereof.

In one example the hollow opening inside the channel is sealed by a rubber element which blocks the passageway through the channel. This rubber element preferably has a resilient lip which is flexible in at least one direction and which thus seals against a part of the injection device. This resilient lip and the part against which the lip seals thus makes up the one-way pressure valve. In one example, the flexible lip seals against a part of the element carrying the cleaning reservoir.

The flexibility of the lip allows passage through the channel and into the overflow reservoir when the pressure inside the cleaning reservoir surpasses a predetermined level defined as the opening pressure.

The cleaning reservoir is distally sealed by a distal septum and proximally sealed by a proximal septum whereas the overflow reservoir distally is sealed by the distal septum and proximally is sealed by the rubber element.

In a further example, the medical injection device comprises a removable protective cap as it is generally known from injection devices. This protective cap preferably covers the part of the injection device carrying the liquid drug. A preferred shape of a medical injection device seems to be that of a fountain pen i.e. an oblong longitudinal pen-shaped device. In such devices is the cartridge containing the liquid drug usually provided at the distal end of the pen-shaped injection device which end is also the end covered by the removable protective cap.

In the above example, the removable protective cap carries the cleaning reservoir contrary to the first example wherein the cleaning reservoir was carried by the axially movable needle shield.

An injection device wherein the cleaning reservoir is carried by the protective cap is known from WO 2014/029018. Herein a cleaning reservoir is provided at a distal end of the protective cap such that the distal tip of the needle cannula is positioned inside the cleaning reservoir when the protective cap is mounted on the injection device, and the distal tip of the needle cannula is positioned outside the cleaning reservoir when the protective cap is removed from the injection device.

However, this known solution has no overflow reservoir connecting to the cleaning reservoir and thus no valve at all.

In a further example, the needle shield is divided into two parts; a first shield part and a second shield part which are axially movable in relation to each other. One of the two parts can then be moved proximally by the protective cap whereas the other part maintains its position. Further, the part moving with the protective shield can be provided with a special tool inserted in the shield part. This special tool thus straightens the distal part of the needle cannula should it be bended during injection.

Regarding the liquid cleaning agent then any liquid substance that are able to clean the distal tip of the needle cannula can be used. The liquid cleaning agent can in one example be filled into the cleaning reservoir by the manufacture of the injection device and delivered to the user in a ready-to-use state.

Alternatively, the user is required to fill the cleaning agent into the cleaning reservoir when taking the injection device into use.

In one example, the liquid pharmaceutical drug contained in the cartridge contains a preservative. In such case, the preservative containing liquid drug can be used as the liquid cleaning agent. This would require the user to transfer a predetermined volume of the preservative containing liquid drug from the cartridge and into the cleaning reservoir. This is preferably done by either moving the plunger forward inside the cartridge or moving the glass part of the cartridge relatively to the plunger. In either way, a predetermined volume is pumped from the cartridge and into the cleaning reservoir.

### Definitions

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel and connected to a hub to form a complete “injection needle” also often referred to as a “needle assembly”. A needle cannula could however also be made from a polymeric material or a glass material. The hub also carries the connecting means for connecting the needle assembly to an injection apparatus and is usually moulded from a suitable thermoplastic material.

As used herein, the term “liquid drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form. The term “preservative containing liquid drug” is preferably used to describe a liquid drug containing a preservative. Such liquid drug could in one example be a blood sugar regulating liquid drug such as insulin, insulin analogue, GLP-1 or GLP-2, and the preservative contained in the liquid drug could in one example be phenol, meta-cresol or any combination thereof.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled injection device” or “Disposable injection device” is meant an injection device containing a predetermined quantum of a liquid drug and which injection device is disposed of once this predetermined quantum has been used. The cartridge containing the liquid drug is permanently positioned or embedded in the injection device such that the user cannot remove the cartridge without permanent destruction of the injection device. Once the predetermined amount of liquid drug in the cartridge and thus in the injection device is used either in one injection or in a series of multiple injections, the user discards the entire injection device including the embedded cartridge.

This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

“Scale drum” is meant to be a cylinder shaped element carrying indicia indicating the size of the selected dose to the user of the injection pen. The cylinder shaped element making up the scale drum can be either solid or hollow. “Indicia” is meant to incorporate any kind of printing or otherwise provided symbols e.g. engraved or adhered symbols. These symbols are preferably, but not exclusively, Arabian numbers from “0” to “9”. In a traditional injection pen configuration the indicia is viewable through a window provided in the housing.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses. Alternatively, the spring can be fully preloaded by the manufacturer with a preload sufficient to empty the entire drug cartridge though a number of doses. Typically, the user activates a latch mechanism e.g. in the form of a button on, e.g. on the proximal end, of the injection device to release—fully or partially—the force accumulated in the spring when carrying out the injection.

The term “Permanently connected” or “permanently embedded” as used in this description is intended to mean that the parts, permanently connected or permanently embedded, requires the use of tools in order to be separated and should the parts be separated it would permanently damage at least one of the parts thereby rendering the construction useless for its purpose.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show an exploded perspective view of a first example of the invention (viewed from a distal position).

FIG. 2 show an exploded perspective view of the first example of the invention (viewed from a proximal position).

FIG. 3 show a side view of the first example of the invention.

FIG. 4 show a perspective view of the movable assembly as seen from a proximal position.

FIG. 5 show a perspective view of the movable assembly as seen from a different position.

FIG. 6 show a cross-sectional view of the first example of the invention with the distal tip of the needle cannula positioned inside the cleaning reservoir.

FIG. 7 show a cross-sectional view of the first example of the invention when rotated 90 degree around the centre axis X relative to FIG. 6.

FIG. 8 show a further cross sectional view of the first example of the invention with the distal tip of the needle cannula positioned outside the cleaning reservoir.

FIG. 9 show a perspective view into the chamber part of the first example of the invention when viewed from a proximal position.

FIG. 10 show a perspective view into the chamber part of the first example of the invention when viewed from a distal position.

FIG. 11 show a cross-sectional view of a second example of the invention.

FIG. 12 show an enlarged view of the distal part of the injection device circled in FIG. 11.

The figures are schematic and simplified for clarity, and they just show details, which are essential to the understanding of the invention, while other details are left out. Throughout, the same reference numerals are used for identical or corresponding parts.

### Detailed Description of Embodiment

When in the following terms as “upper” and “lower”, “right” and “left”, “horizontal” and “vertical”, “clockwise” and “anti (or counter) clockwise” or similar relative expressions are used, these only refer to the appended figures and not to an actual situation of use. The shown figures are schematic representations for which reason the configuration of the different structures as well as their relative dimensions are intended to serve illustrative purposes only.

In that context it may be convenient to define that the term “distal end” in the appended figures is meant to refer to the end of the injection device which usually abut or points towards the skin of the user during injection and which end carries the injection needle whereas the term “proximal end” is meant to refer to the opposite end pointing away from the skin during injection and which is often provided with a rotational button to set the size of the dose to be injected. Distal and proximal is meant to be along an axial orientation of the injection device along a virtual centre line marked “X” in FIG. 3.

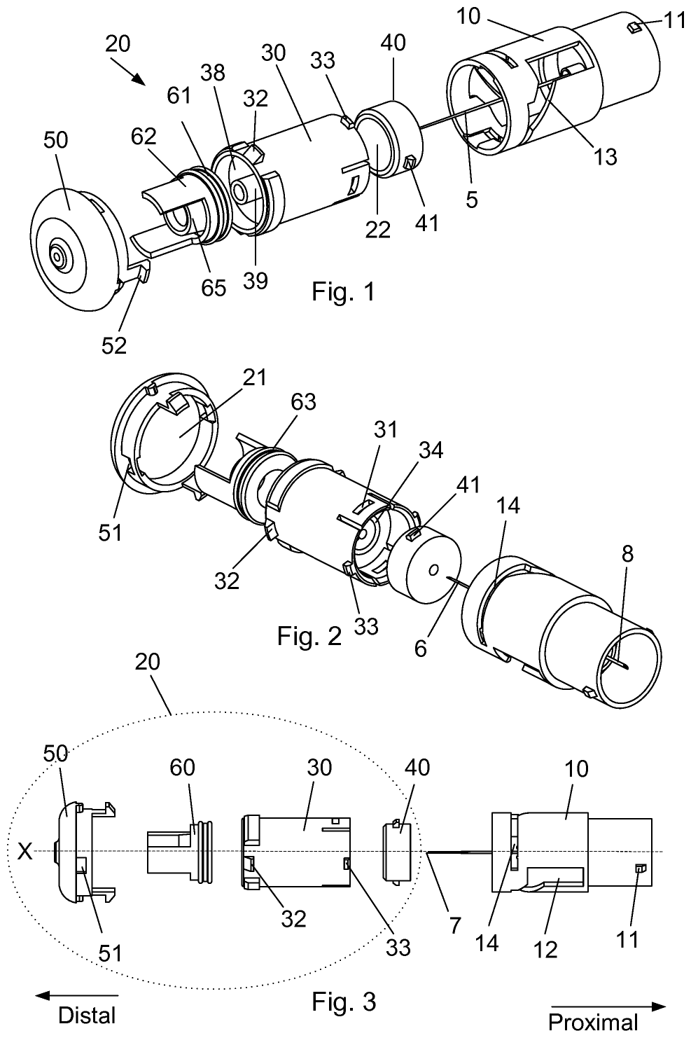

FIGS. 1 and 2 each disclose a perspective exploded view of the front end of the injection device according to a first embodiment whereas FIG. 3 is an exploded side view.

The depicted front end comprises a stationary part 10 and a movable assembly 20. The stationary part 10 is a part of the housing structure of the injection device and is secured to the remaining housing structure by having a radial protrusion 11 which is click fitted to the remaining housing structure. The movable assembly 20 is indicated with a broken line in FIG. 3.

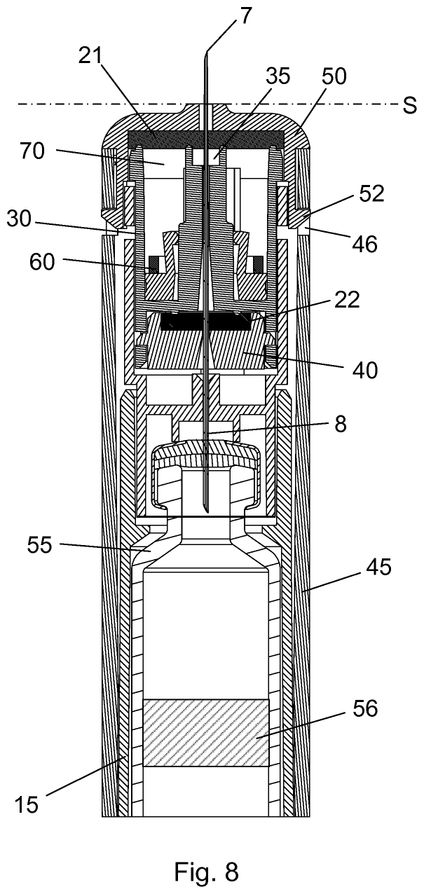

The remaining housing structure includes a cartridge holder 15 as best seen in FIG. 8. The cartridge holder 15 secures the cartridge 55 which contains the liquid drug to be injected as it is commonly known from injection devices. The injection itself is performed by moving a plunger 56 forward inside the cartridge 55 thus creating an overpressure inside the cartridge 55.

The stationary part 10 is further provided with a longitudinal track 12 for guiding the movable assembly 20 via a guiding protrusion 33 provided on the movable assembly 20. In the depicted embodiment, the guiding protrusion 33 is provided on a chamber part 30.

The longitudinal track 12 connects to a radial track 14 as e.g. seen in FIGS. 2 and 3. When the guiding protrusion 33 is positioned inside this radial track 14, the movable assembly 20 is prevented from moving in the axial direction. A relative rotation between the movable assembly 20 and the stationary part 10 thus unlocks the injection device and makes it possible to move the movable assembly 20 axially relatively to the stationary part 10.

In the disclosed example, the axial track 12 connects to the radial track 14 via a helical track 13. When using such a helical track 13, the movable assembly 20 can be moved a predetermined distance in the axial direction upon rotation. However, the axial track 12 could easily connect directly to the radial track 14.

The stationary part 10 also carries a needle cannula 5 which has a distal part 6 which extend in the distal direction and a proximal part 8 which extend in the proximal direction. The proximal part 8 is usually connected to a reservoir such as a cartridge 55 containing the liquid drug to be injected whereas the distal part 6 is provided with a sharp tip 7 for penetrating the skin of the user during injection. Stretching in the longitudinal direction along the axis X, the needle cannula 5 is provided with a hollow lumen 9 through which the liquid drug flows during injection.

Since the stationary part 10 carries the needle cannula 5 it could also be denoted as the hub when comparing to well-known needle assemblies.

The movable assembly 20 comprises a chamber part 30 sandwiched between a bottom part 40 and a top part 50 as best seen in FIG. 3.

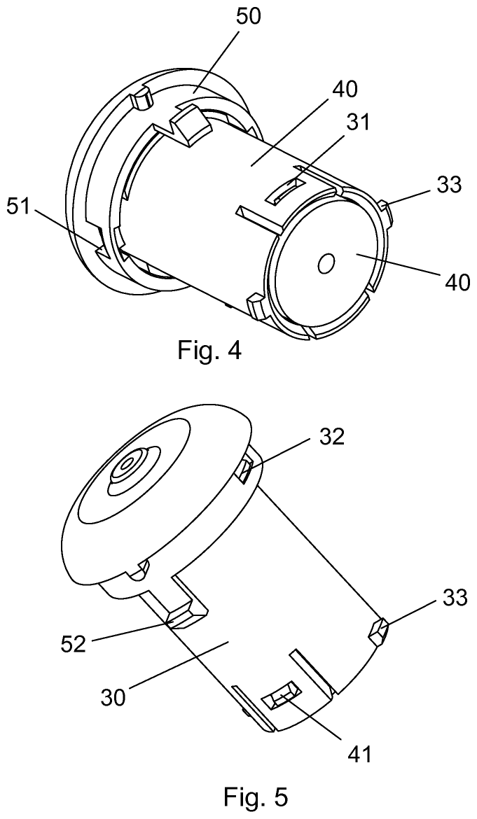

Proximally, the chamber part 30 is provided with a number of radially extending chamber part openings 31 into which radially extending bottom part protrusions 41 on the bottom part 40 clicks during assembly of the movable assembly 20. In the same fashion, outwardly extending chamber part protrusions 32 provided on the chamber part 30 clicks into top part openings 51 provided in the top part 50 such that the chamber part 30, the bottom part 40 and the top part 50 can be click fitted together to form one cleaning assembly as disclosed in FIGS. 4 and 5.

As seen in FIG. 8, the movable assembly 20 can be connected to a telescopically movable needle shield 45 to move with the needle shield 45. The top part 50 is henceforth provided with outwardly pointing shield protrusions 52 for this purpose, and the needle shield 45 is provided with radial openings 46 for receiving these outwardly pointing shield protrusions 52. The movable assembly 20 thus moves telescopically together with the needle shield 45.

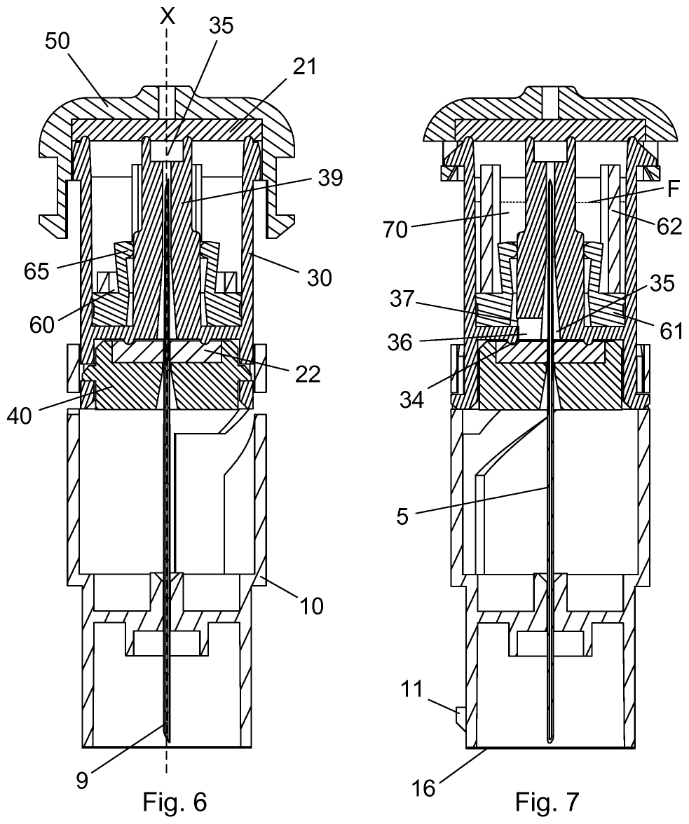

The combined unit 10, 20 disclosed in FIGS. 6 and 7 can be sterilized as one unit and for that purpose the combined unit 10, 20 is sealed by a pierceable foil 16 provided distally on the stationary part 10. This foil 16 is removed or broken when the distal part 8 of the needle cannula 5 is penetrated into the cartridge 55 as disclosed in FIG. 8.

As best seen in the FIGS. 6 to 8 (and in FIG. 1-2), a distal septum 21 is provided between the chamber part 30 and the top part 50, and a proximal septum 22 is provided between the chamber part 30 and the bottom part 40.

The distal septum 21 is moulded as an integral part of the top part 50 and the proximal septum 22 is moulded to form an integral part of the bottom part 40 as disclosed in FIGS. 1 and 2. The septae 21, 22 are preferably moulded from a suitable rubber material (TPE) whereas the bottom part 40 and the top part 50 are moulded from a suitable polymer. The moulding technique used could be a so-called multi-component moulding using different materials supplied through different injection points in a sequential injection moulding process, such multi-component moulding are often referred to as 2K moulding. However, the two septae 21, 22 could alternatively be provided as individual septae sandwich in their respective positions.

Further, the septae 21, 22 are pierceable in a self-sealing manner as generally known from septae used in medical containers, and both the top part 50 and the bottom part 40 are provided with openings for passage of the needle cannula 5.

The chamber part 30 has a through-going opening which when closed by the distal septum 21 and the proximal septum 22 makes up a cleaning reservoir 35. Between subsequent injections, the distal tip 7 of the needle cannula 5 is maintained inside this cleaning reservoir 35 as disclosed in FIGS. 6 and 7.

The proximal end surface of the chamber part 30 is provided with an axially extending circular protrusion 34 which seals against the proximal septum 22 as best seen in FIGS. 6 and 7.

Further, in the centre of the chamber part 30 and surrounding the cleaning reservoir 35, a tower 39 is erected which distally seals against the distal septum 21. The cleaning reservoir 35 is thus tightly sealed at both ends.

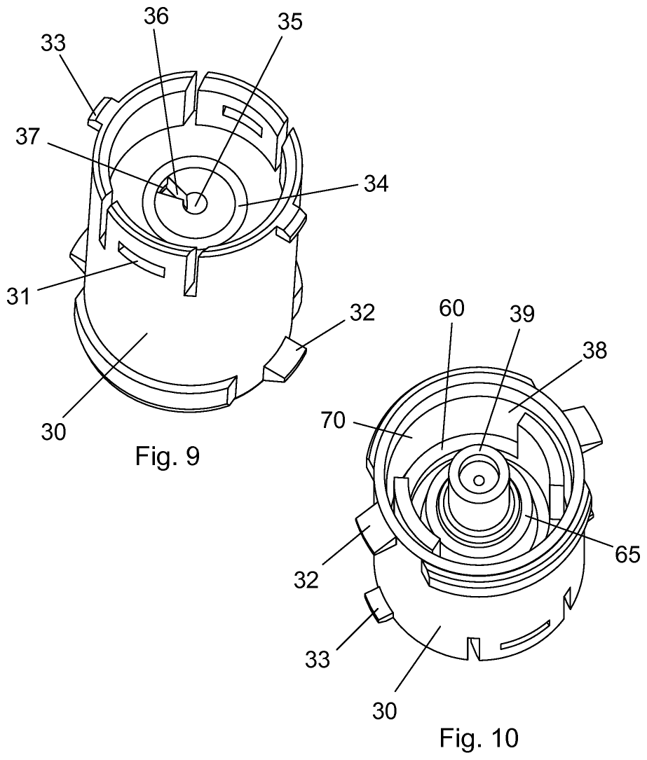

FIG. 9 discloses a view of the chamber part 30 as seen from the proximal end of the injection device and shows that the cleaning reservoir 35 is provided with a large radial opening 36 which connects to a continued small radial opening 37 (as also seen in FIG. 7). This small radial opening 37 terminates into a distal space 38 of the chamber part 30 as best seen in FIG. 1 and in FIG. 10. However, in FIG. 10, the sealing part 60 is located inside the distal space 38.

The design of a large radial opening 36 continuing into a small radial opening 37 is mainly due to the moulding technique used. During moulding one core can thus be used to make the large distal space 38 and a second core can be used to form the radial opening 36. The small radial opening 37 will thus arise where the two cores abut.

As best seen in FIGS. 1 to 3, the distal space 38 of the chamber part 30 is occupied by a sealing part 60. This sealing part 60 is a two component part and comprises a flexible rubber (TPE) part 61 and a more rigid plastic part 62. The flexible rubber part 61 and the plastic part 62 are preferably made by 2K moulding, but could alternatively be two separate parts joined together e.g. by gluing.

The flexible rubber part 61 is on the outer surface provided with an outer seal 63 which seals against the inner surface of the chamber part 30. This outer seal 63 is in the disclosed embodiment formed as two circular ridges or crests as e.g. disclosed in FIG. 2. On the inner surface, the flexible rubber part 61 seals against the tower 39 of the chamber part 30 preferably through a circular lip 65.

This circular lip 65 is inclined towards the tower 39 with an inherent resiliency such that the circular lip 65 and the tower 39 together form a valve only allowing passage of the liquid drug when the pressure is able to open the valve i.e to force the circular lip 65 radially away from the outer surface of the tower 39.

The part of the space 38 defined by the interior of the chamber part 30, the circular lip 65 and the distal septum 21 makes up the overflow reservoir 70.

In case the liquid drug contained in the cartridge 55 also comprises a preservative, this preservative containing liquid drug can be used as a cleaning agent. This would require the user to eject a volume of the preservative containing liquid drug from the cartridge 55 and into the cleaning reservoir 35. When the preservative containing liquid drug is ejected from the cartridge 55 and into the cleaning reservoir 35, the air inside the cleaning reservoir 35 will be forced out through the valve and into the overflow reservoir 70. At the end of filling liquid drug into the cleaning reservoir 35, a small volume of the liquid drug also will pass through the radial openings 36, 37 and pass the circular lip 65 and is thus pressed into the overflow reservoir 70. This is indicated by the line “F” in FIG. 7.

In FIG. 7, the cleaning reservoir 35 is shown as being filled with the same preservative containing liquid drug as present in the cartridge 55. Further, a volume of the preservative containing liquid drug has flown through the valve and occupies a part of the overflow reservoir represented by the line “F”. Proximally to the line “F”, the air is now compressed.

Since the air contained in the proximal part of the overflow reservoir 70 is compressed, this air applies a pressure on the preservative containing liquid drug which helps urging the inclined circular lip 65 towards the tower 39. The valve thus operates as a one-way valve only allowing passage from the cleaning reservoir 35 and into the overflow reservoir 70.

Ejecting preservative containing liquid drug into the cleaning reservoir 35 is done by moving the plunger 56 and the cartridge 55 relatively to each other, preferably by either moving the plunger 56 distally or moving the cartridge 55 itself proximally or both. This builds up a pressure in the preservative containing liquid drug inside the cartridge 55 and the drug flows through the lumen 9 of the needle cannula 5 and into the cleaning reservoir 35 and further out through the one-way valve and into the overflow reservoir 70 until the pressure in the compressed air equalizes the pressure inside the cartridge. Once this balance has been reached, the preservative containing liquid drug stop to flow from the cartridge 55 and into the cleaning reservoir 35.

FIG. 10 discloses a view inside the chamber part viewed from a distal position. As seen segments of the rigid plastic part 62 of the sealing member 60 has been cut away to allow the interior space 38 of the chamber part 30 to have a larger volume.

FIG. 8 discloses the injection device during injection. The movable assembly 20 together with the needle shield 45 has been moved to a proximal position preferably by pressing the top part 50 against the skin of the user as indicated by the line “S” in FIG. 8. During injection, the plunger 56 is moved forward inside the cartridge 55 preferably by an automatic drive mechanism as generally known. Following the injection, when the user removes the injection device from the skin, a not-shown spring element moves the movable assembly 20 in the distal direction to the position disclosed in FIGS. 6 and 7 wherein the distal tip 7 of the needle cannula 5 is positioned inside the cleaning reservoir 35.

### Second Embodiment—Reservoir in Cap

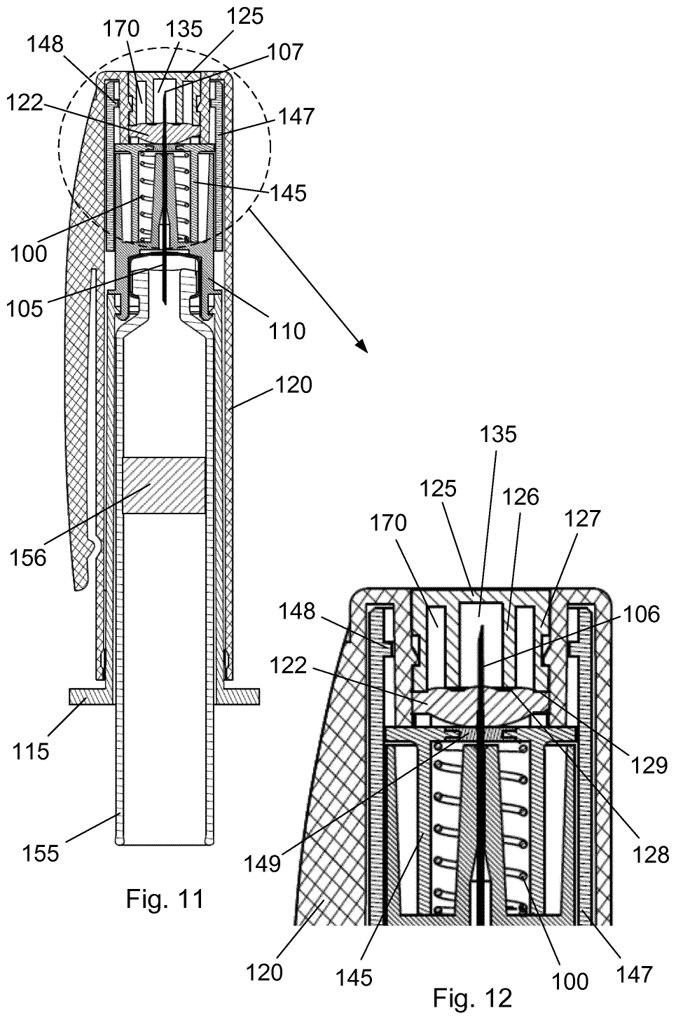

FIGS. 11 and 12 discloses a different embodiment wherein the same or similar elements are numbered with the same numbers as in the first embodiment, however, with a “1” in front. The needle cannula which in the first embodiment is numbered 5 is the second embodiment numbered 105.

The needle cannula 105 has a distal tip 107 which penetrates the skin of the user during injection and a proximal part 108 which is inserted into a cartridge 155 such that liquid drug can flow from the cartridge 155, through the lumen of the needle cannula 105 and through the skin of a user.

In this second embodiment, the cleaning reservoir 135 is carried by a removable protective cap 120. This protective cap 120 is releasable mounted on the housing structure as is commonly known form injection devices.

As depicted in FIG. 11, the stationary part 110 carrying the needle cannula 105 is click-fitted to the cartridge holder 115 and is as such a part of the housing structure.

As also disclosed in the first embodiment, the needle cannula 105 is anchored in the stationary part 110 which thus operates a hub in a needle assembly.

The removable protective cap 120 is distally provided with an insert 125. This insert has a circular inner wall 126 and a circular outer wall 127. Both these circular walls 126, 127 stretch in the proximal direction.

The space encompassed by the circular inner wall 126 is the cleaning reservoir 135 and the space located between the circular inner wall 126 and the circular outer wall 127 is the overflow reservoir 170.

Proximally the circular inner wall 126 terminates in an inner wall end 128 whereas the circular outer wall 127 terminates in an outer wall end 129. Both these ends 128, 129 abut a proximal cap septum 122.

The proximal cap septum 122 is press fitted into the insert 125 such that the connection between the outer wall end 129 and the proximal cap septum 122 is fluid tight whereas the connection between the inner wall end 128 and the proximal cap septum 122 is able to open when the pressure inside the cleaning reservoir 135 surpasses a certain level.

The connection between the inner wall end 128 and the cap septum 122 in combination form a pressure valve which opens at a certain and predetermined pressure.

When preservative containing liquid drug is transferred from the cartridge 155 and into the cleaning reservoir 135, the air contained in the cleaning reservoir 135 is pressed through the pressure valve 122, 128 and into the overflow reservoir 170. If the cleaning reservoir 135 is overfilled, a surplus of preservative containing liquid drug also flows into the overflow reservoir 170. The air previously contained inside the overflow reservoir 170 is henceforth compressed and pressurized during the filling of the cleaning reservoir 135.

The needle shield is in this second example divided into two parts. A first shield part 145 and a second shield part 147. The first shield part 145 is movable in the proximal direction against the bias of a compression spring 100 as disclosed in FIGS. 11 and 12.

When the user wants to perform an injection, the user removes the protective cap 120 whereby the spring element 100 moves the first shield part 145 distally and into alignment with the second shield part 147. When the protective cap 120 has been removed, the distal end of the first shield part 145 thus abuts an inwardly pointing ring-shaped ridge 148 provided on the second shield part 147.

During injection, the user pushes the second shield part 147 against the skin and the first shield part 145 follows in the proximal direction as the distal tip 107 of the needle cannula 105 penetrates the skin of the user.

The injection itself is preferably done automatically by a spring driven injection mechanism as it is commonly known.

Following an injection, when the user removes the distal end of the injection device from the skin, the spring element 100 urges the first shield part 145 and therewith also the second shield part 147 in the distal direction.

Following the injection, when the user remounts the protective cap 120 on the injection apparatus, the protective cap 120 pushes the first shield part 145 in the proximal position into the position depicted in FIG. 11. In this position, the distal tip 107 of the needle cannula 105 is once again positioned inside the cleaning reservoir 135.

The first shield part 145 is in a further embodiment distally provided with an insert tool 149. This insert tool 149 is made from a different material (e.g. TPE) and is in one example 2K moulded together with the first shield part 145. The purpose of this insert tool 149 is to realign the needle cannula 105 should it somehow be bended during use.

During use it sometimes happens that the distal part 106 of the needle cannula 105 gets bended a little such that the distal part 106 of the needle cannula 105 deviates from the centre line X. This could potentially create problems when the distal tip 107 of the needle cannula 105 has to re-enter the cleaning reservoir 135. In worst case, the distal part 106 of the needle cannula 105 could be bended to a degree wherein the distal tip 107 do not re-enter the cleaning reservoir 135. In order to overcome this, the first shield part 145 is preferably provided with an insert tool 149 which will straighten out the distal part 106 of the needle cannula 105 during movement of the first shield part 145 and thus the insert tool 149 in the distal direction as it occurs when the second shield part 147 is removed from the skin of the user following an injection.

Once the distal part 106 is straight and aligned with the centre axis X, the distal tip 107 of the needle cannula 105 slides directly into the cleaning reservoir 135 when the protective cap 120 is re-mounted on the injection device.

Some preferred embodiments have been disclosed in the foregoing, but it should be stressed that the invention is not limited to these, but may be embodied in other ways within the subject matter defined in the following claims.

1. A medical injection device for injection of a liquid drug, comprising:
a housing structure supporting a cartridge containing the liquid drug, wherein the cartridge comprises a plunger which is movable in a distal direction inside the cartridge to thereby pressurize the liquid drug contained in the cartridge,
a needle cannula having a distal part with a distal tip and a longitudinal lumen, the needle cannula being mounted relatively to the housing structure such that the distal tip extend in a distal direction and wherein the needle cannula proximally connects to the cartridge at least during injection,
an axially movable cleaning reservoir movable in relation to the housing structure, and which cleaning reservoir connects to an overflow reservoir and contains a liquid cleaning agent, and wherein the distal tip of the needle cannula is positioned inside the cleaning reservoir at least between injections,
wherein an overflow reservoir is connected to the cleaning reservoir through a one-way pressure valve comprising a flexible element changeable from a default closed state to an activated open state,
and wherein the flexible element in the default closed state seals off the cleaning reservoir and in the activated open state allow the cleaning agent and/or air to flow from the cleaning reservoir and into the overflow reservoir,
and which flexible element is changeable from the default closed state to the activated open state when the pressure inside the cleaning reservoir surpasses a predetermined level.

2. The medical injection device according to claim 1, wherein the injection device further comprises an axially movable needle shield which is movable between a first position and a second position, wherein the needle shield in the first position covers the distal tip of the needle cannula, and
in the second position is retracted to a position wherein the distal tip of the needle cannula extend distally from the needle shield.

3. The medical injection device according to claim 2, wherein the needle shield carries the cleaning reservoir, such that
the distal tip of the needle cannula is positioned inside the cleaning reservoir when the needle shield is in the first position, and wherein the distal tip of the needle cannula extend distally in relation to the cleaning reservoir when the needle shield is retracted to the second position.

4. The medical injection device according to claim 1, wherein the cleaning reservoir and the overflow reservoir is connected by a channel.

5. The medical injection device according to claim 4, wherein the channel is blocked by a flexible element having a flexible lip with an inherent resiliency.

6. The medical injection device according to claim 5, wherein the flexible lip has a circular cross section and radially abut a tower centrally located in the cleaning reservoir.

7. The medical injection device according to claim 1, wherein the cleaning reservoir distally is sealed by a distal septum and proximally is sealed by a proximal septum.

8. The medical injection device for apportioning set doses according to claim 7, wherein the overflow reservoir distally is sealed by the distal septum and proximally is sealed by the flexible element.

9. The medical injection device according to claim 2, wherein the injection device comprises a removable protective cap.

10. The medical injection device according to claim 9, wherein the removable protective cap carries the cleaning reservoir, such that the distal tip of the needle cannula is positioned inside the cleaning reservoir when the protective cap is mounted on the injection device, and the distal tip of the needle cannula is positioned outside the cleaning reservoir when the protective cap is removed from the injection device.

11. The medical injection device according to claim 10, wherein the movable needle shield comprises a first shield part and a second shield part which are axially movable in relation to each other.

12. The medical injection device according to claim 1, wherein the cleaning agent is identical to the liquid drug contained in the cartridge and wherein the liquid drug contains a preservative.

13. The medical injection device according to claim 12, wherein a predetermined volume of the preservative containing liquid drug is transferable from the cartridge and into the cleaning reservoir.

14. The medical injection device according to claim 13, wherein the pressure inside the cleaning reservoir surpasses a predetermined level when the preservative liquid drug is transferred from the cartridge and into the cleaning reservoir.
